(19) United States
(12) Patent Application Publication
(10) Pub. No.: US 2016/0220373 A1
(43) Pub. Date: Aug. 4, 2016

(54) PENILE PROSTHETIC ASSEMBLY HAVING SELECTIVELY CONTROLLED VIBRATORY UNIT

(71) Applicants: Felix Oben, Fulton, NY (US); Gary L. Lagler, Mexico, NY (US); Alfred S. Greco, Fulton, NY (US)

(72) Inventors: Felix Oben, Fulton, NY (US); Gary L. Lagler, Mexico, NY (US); Alfred S. Greco, Fulton, NY (US)

(21) Appl. No.: 15/012,164

(22) Filed: Feb. 1, 2016

Related U.S. Application Data
(60) Provisional application No. 62/161,643, filed on May 14, 2015.
(60) Provisional application No. 62/125,797, filed on Feb. 2, 2015.

(51) Int. Cl. A61F 2/26; A61H 19/00

(52) U.S. Cl. CPC A61F 2/26; A61H 19/50

(57) ABSTRACT

A penile prosthetic assembly includes at least one cylinder made from a medical grade material that is configured for surgical implantation within the corpora chambers of a penis. A vibratory unit attached to the at least one cylinder includes a motor connected to a portable power source, such as at least one battery. A control mechanism, such as an implantable switch or wireless remote control unit, selectively enables the vibratory unit which can include various vibratory modes. The cylinders of the penile prosthetic assembly can either be inflatable or malleable versions.

### CROSS REFERENCE TO RELATED APPLICATIONS

This application claims priority under applicable sections of 35 USC §111 and 37 CFR §1.53 to U.S. Application Ser. No. 62/161,643, filed May 14, 2015 and entitled: PENILE PROSTHETIC ASSEMBLY HAVING SELECTIVELY CONTROLLED VIBRATORY UNIT, and U.S. Application Ser. No. 62/125,797, filed Feb. 3, 2015, and entitled: ERECTILE DEVICE. The contents of each of these applications is herein incorporated by reference in their entirety.

### TECHNICAL FIELD

This application generally relates to the field of prosthetic devices and more specifically to a penile prosthetic assembly having a set of surgically implantable cylinders, including an integrated or attached vibratory unit(s) that is selectively engageable by a user, for example, for the treatment of severe erectile dysfunction (ED). The vibratory unit can be used in conjunction with malleable (non-inflatable) and/or inflatable cylinders that are used to produce an erection-like state and in which the integrated or attached vibratory unit can be controlled by a wired or a wireless connection.

### BACKGROUND

As is known, the treatment of erectile dysfunction (ED) is caused by the inability of a male adult to maintain an erection that is sufficient for sexual activity. An initial treatment for this condition may be prescribed by a physician or other caregiver in the form of an oral prescriptive mediation such as Viagra (Sildenafil citrate), Cialis (Tadalafil), Levitra (Vardenafil HCl) or other vasodilatory drugs. If these treatments are ineffective or unsuccessful, then other options may be considered. These options may include use of a vacuum erectile device, intra-urethral medications, or penile injection therapy. If these latter options also are unsuccessful then a further treatment option, penile prosthesis implantation, may be considered.

Penile prosthetic devices or assemblies are completely implanted surgically within the body of a patient. The function of the penile prosthetic device is to produce an erection-like state that enables a man to have normal sexual activity. Neither the operation to implant the prosthesis nor the apparatus itself will interfere with the patient's sensation, orgasm, ejaculation or urination.

More specifically, there are two erection chambers (corpora cavernosa) located within the penis. A penile prosthetic device typically includes flexible cylinders made from a medical grade material (silicone) that are capable of producing a rigidity or firmness to enable the man to have sexual intercourse when the cylinders are disposed within the corpora chambers. The types of prosthetic devices can include malleable versions or inflatable versions. Inflatable penile prostheses are fluid-filled devices that can be utilized for erection that allow for control of rigidity and size, in a manner that is more natural akin to a normal penis.

There is a continuing and general need to improve upon penile prosthetic apparatus in this field.

### BRIEF DESCRIPTION

According to one aspect, there is described a penile prosthetic assembly comprising at least one cylinder made from a medical grade material that is configured for surgical implantation within the corpora chambers of a penis. A vibratory unit is attached and operatively connected to the at least one cylinder, the vibratory unit including a motor connected to a portable power source. A control mechanism selectively operates the vibratory unit.

In one version, the control mechanism is an actuable control that is surgically implanted and made accessible to the patient. According to another version, a control unit can be wirelessly connected to the penile prosthetic assembly, and more specifically the vibratory unit, for enabling remote operation.

When the vibratory unit is activated by the user, the implanted cylinders and therefore the entire penis is caused to vibrate, adding to the sensation of the partner during intercourse.

According to another version, the implanted cylinders are hollow and made from a flexible medical grade material. The cylinders are connected to a fluid source such as a fluid reservoir, wherein the prosthetic assembly further includes a pump that is connected to a portable power source such as batteries. The pump is configured to selectively pump contained fluid from the fluid reservoir into the interior of the cylinders. In this version, the control mechanism can further include at least one implantable switch or alternatively, a remote control unit, in order to selectively energize and deenergize the pump and/or the vibratory unit.

According to another version, there is provided a penile prosthetic assembly comprising a pump, a reservoir containing a volume of fluid, and a set of cylinders fluidically connected to the pump and reservoir. The cylinders, pump and reservoir are sized for implantation within the penis and configured such that fluid can be pumped into and out of the cylinders to enable inflation and deflation thereof. A vibratory unit is attached and operatively connected to the cylinders, wherein the assembly further includes a portable power source preferably connected to the pump and the vibratory unit. A control mechanism selectively controls the operation of the vibratory unit and the pump.

In one version, the vibratory unit includes a motor disposed upon each of the flexible hollow cylinders which is connected to the portable power source, e.g., a battery. The vibratory unit can be controlled by an actuable switch that is surgically implanted and otherwise is accessible to the patient. According to another version, a control unit is wirelessly connected to the prosthetic assembly to enable remote operation.

One advantage provided by the herein described assembly is that of providing a further improvement in the treatment of severe erectile dysfunction.

These and other features and advantages will be readily apparent to one of sufficient skill from the following Detailed Description, which should be read in conjunction with the accompanying drawings.

### BRIEF DESCRIPTION OF THE DRAWINGS

FIG. 1 depicts a perspective view of a penile prosthetic assembly made in accordance with an embodiment of the present invention; and

FIG. 2 depicts a perspective view of a penile prosthetic assembly made in accordance with another embodiment in accordance with the present invention.

### DETAILED DESCRIPTION

The following relates to a penile prosthetic assembly made in accordance with various embodiments. It will be readily apparent, however, that other variations and modifications can be made within the spirit and scope of this description. In addition, various terms are used throughout the description in order to provide an adequate frame of reference with regard to the accompanying drawings. These terms, unless so specifically indicated, should not be regarded as overly limiting in terms of the scope of the herein described prosthetic apparatus. In addition, the drawings are intended to adequately depict sufficient details of the apparatus. To that end, the drawings should not be relied upon for scaling purposes.

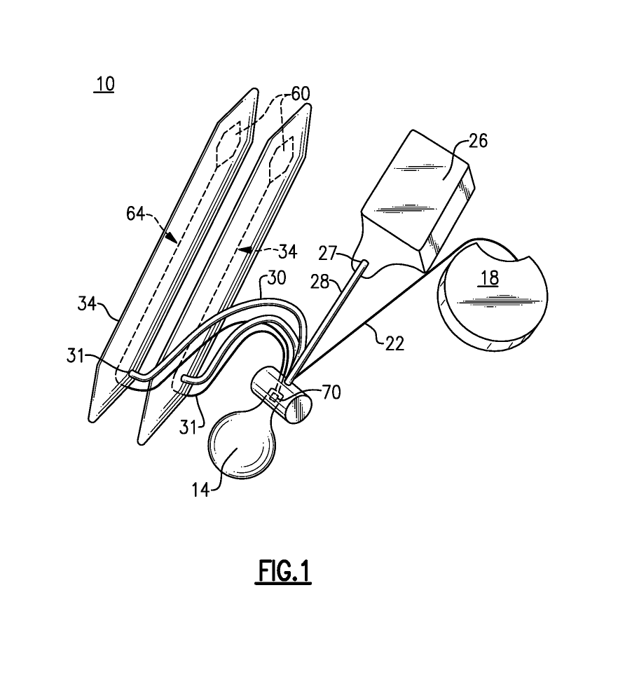

Referring to FIG. 1, a penile prosthetic assembly 10 is provided in accordance with a first embodiment. The penile prosthetic assembly 10 is defined by a plurality of interconnected components including a compact pneumatic pump 14 that is connected to a portable power source, such as a battery 18, through a corresponding set of electrical leads 22. The pump 14 is manufactured from a biocompatible material, such as silicone, and can include a reciprocating piston or other suitable element that is disposed within a pump housing. Alternatively, the pump can be made in accordance with other suitable designs that are capable of pumping a fluid. According to this specific embodiment, the pump 14 is configured to direct fluid from the confines of a sealed reservoir 26 to and from a set of flexible hollow cylinders 34. The sealed reservoir 26 contains a volume of fluid that can be accessed through a port 27 at one end of the reservoir 26 by an extending end of a flexible fluid conduit or hose 28, the latter being connected to respective inlet and outlet ports of the pump 14. According to this embodiment, the contained fluid is saline, although other suitable bio-compatible fluids can be alternatively be used.

Each of the pair of hollow cylinders 34 are fluidically connected to the compact pump 14 by means of a corresponding pair of hoses 30, allowing fluid to be pumped into and from each cylinder 34 and to and from the reservoir 26 via the conduit 28. Each of the cylinders 34 is defined by an elongate configuration, including a port 31 disposed at one end thereof. The port 31 is configured to receive an end of the hose 30 to permit fluid from the sealed reservoir 26 to be selectively pumped into and out of the interior of each cylinder 34. The cylinders 34 are further defined by a hollow interior according to this embodiment, wherein each of the cylinders 34 and hoses 28, 30 are fabricated from a medical grade material, such as silicone. It will be apparent that other suitable materials can be utilized provided these materials enable inflation of the hollow cylinders 34 through fluid expansion of same.

Still referring to FIG. 1 and according to this exemplary embodiment, each of the flexible hollow cylinders 34 of the prosthesis 10 further includes a vibratory unit 60 having a set of extending electrical leads 64. More specifically, each of the vibratory units 60 comprise a motor, although any suitable device that is capable of producing a vibratory or oscillatory effect can be alternatively provided. As to attachment, the vibratory units 60 can be disposed within a compartment formed within the interior of the cylinder 34 or alternatively, the vibratory units 60 can be disposed on the exterior of the cylinders 34 and secured by suitable means, such as adhesives. According to this specific embodiment, the vibratory units 60 are commonly located at one end of the cylinders 34, but it will be readily apparent that the units 60 can be disposed upon any suitable portion thereof. According to this specific embodiment, each of the electrical leads 64 extending from the vibratory units 60 are further connected to an actuable switch 70 and the battery 18 that enables selective energization of the vibratory motors 60 via a portable power source (battery) 18. Alternatively, the units 60 could contain their own independent power source in lieu of the battery 18. Similar control means (not shown) are provided for selectively energizing the pump 14 to enable inflation of the cylinders 34.

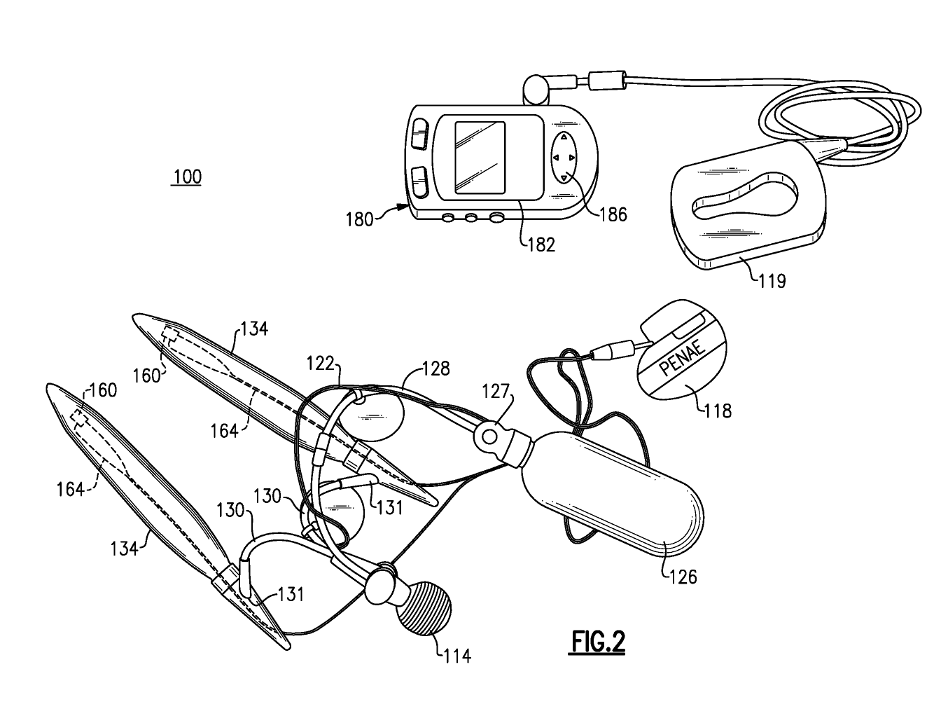

In use, the herein described prosthetic assembly 10 can be surgically implanted with the cylinders 34 being implanted within the corpora chambers of the penis of the patient. The reservoir 26 and battery 18 are preferably implanted in the retropubic space and the pump 14 of the assembly 10 is preferably implanted within the patient's scrotom. Prior to assembly, a cover 119, FIG. 2, is placed onto the battery 18.

According to this embodiment, the actuable switch 70 is disposed in relation to the pump 14 and is accessible to the patient following implantation to enable selective energization of the vibratory units 60. When the actuable switch 70 is engaged, the vibratory units 60 cause the cylinders 34 to vibrate, which further causes the entire penis to vibrate as well, thereby creating stimulation. The actuable switch 70 may include one or multiple settings, enabling the vibratory rate to be adjusted.

A penile prosthetic assembly 100 in accordance with another embodiment is shown in FIG. 2 prior to its implantation. Similarly, this prosthetic assembly 100 is defined by a plurality of interconnected components including a compact pneumatic pump 114 that is connected to a portable power source, such as at least one battery 118, through a corresponding set of electrical leads 122. The pump 114 is manufactured from a biocompatible material, such as silicone, and can include a reciprocating piston or other suitable element or can be made in accordance with other suitable designs that are capable of pumping a fluid. According to this exemplary embodiment, the pump 114 is configured to direct fluid from the confines of a sealed reservoir 126 to and from a set of flexible hollow cylinders 134. As in the preceding embodiment, the reservoir 126 contains a volume of fluid that can be accessed through a port 127 at one end of the reservoir 126 to an extending end of a flexible fluid conduit or hose 128 connected to the inlet and outlets of the pump 114. According to this embodiment, the contained fluid is saline, although other suitable fluids can alternatively be used.

Each of the pair of hollow cylinders 134 are fluidically connected to the compact pump 114 by means of a corresponding pair of hoses 130. Each of the cylinders 134 is defined by an elongate configuration, including a port 131 disposed at one end thereof. The port 131 is configured to receive an end of the hose 130 to permit fluid from the reservoir 126 to be selectively pumped into and out of the interior of each cylinder 134. The cylinders 134 are further defined by a hollow interior according to this embodiment, wherein each of the cylinders 134 and hoses 128, 130 are fabricated from a medical grade material, such as silicone. It will be apparent that other suitable materials can be utilized provided they enable inflation of the hollow cylinders 134 through fluid expansion of same.

Still referring to FIG. 2 and according to this specific embodiment, each of the flexible hollow cylinders 134 of the prosthesis 10 further includes a vibratory unit 160 having a set of extending electrical leads 164. More specifically, each of the vibratory units 160 comprise a motor, although any suitable device capable of producing a vibratory or oscillatory effect can be provided. As to attachment, the vibratory units 160 can be disposed within a compartment formed within the interior of the cylinder 134 or alternatively the vibratory units 160 can be disposed on the exterior of the cylinders 134 and secured by suitable means, such as adhesives. According to this specific embodiment, the vibratory units 160 are commonly located at one end of the cylinders 134, but it will be readily apparent that the units 160 can be disposed along any suitable portion thereof. According to this specific embodiment, each of the electrical leads 164 extending from the vibratory units 160 are further connected to a suitable portable power source (e.g., the battery 118) that enables selective energization of the vibratory units 160. Alternatively and though not shown, the vibratory units 160 can include an independent discrete power source. The vibratory unit 160 can be configured to provide pulsations over varying durations and cycles, thereby comprising various operating modes.

In lieu of an actuable switch and according to this embodiment, a remote control unit 180 can be connected, preferably in a wireless manner such as Bluetooth, Zigbee or other local RF or IR wireless protocol, to the herein described assembly 100 wherein the control unit includes a corresponding receiver/transmitter and optionally a small microcontroller that is used to transmit control signals to activate the pump 114 or vibratory unit(s) 160. The implanted assembly 100 further includes a receiver that receives the at least one RF or IR (or other wireless signal) in which the received signal is used in order to engage or disengage operation of the pump 114 and/or the vibratory unit 160. According to this embodiment, the remote control unit 180 is defined by a housing 182 having a plurality of actuable buttons 186 that define a user interface and in which the operation of each of the inflation and vibratory functions (e.g., pulsatile or other modes) can be enabled selectively by the patient remotely.

In use and according to the embodiment shown in FIG. 2, the assembly 100 is surgically implanted with the exception of the remote control unit 180. Following implantation, the control unit 180 can be then used to selectively operate either the inflation of the hollow cylinders 134 and/or activate the vibratory units 160 of the implanted prosthetic assembly 100. Separate controls 186 can be provided for each function on the user interface of the housing of the remote control unit 180. Alternatively, either the vibratory units 160 and/or the pump 114 can be operated such as using an implanted switch 70, FIG. 1.

Preferably, the portable battery 18, 118 is analogous to those used in other implantable devices, such as pacemakers and sacral neuromodulators, and designed with a working life lasting upwards of five (5) years with continuous use. If replacement of the portable battery 18, 118 is required, a minor surgical procedure would involve a small incision in the retropubic space in which the battery 18, 118 is preferably implanted, as noted above.

### PARTS LIST FOR FIGS. 1-2

10 penile prosthetic assembly

14 pump

18 battery

19 battery cover

22 electrical leads

26 reservoir, fluid

27 port, reservoir

29 hose or conduit

30 hoses

31 port, cylinder

34 set of hollow flexible cylinders

60 vibratory unit

64 electrical leads

70 switch

100 penile prosthesis assembly

114 pump

118 battery

119 battery cover

122 electrical leads

126 reservoir, fluid

127 port, reservoir

129 hose or conduit

130 hoses

131 port, cylinder

134 set of hollow flexible cylinders

160 vibratory unit

164 electrical leads

180 remote control unit

182 housing

186 buttons, actuable

The herein described embodiments are intended to be examples and it should be apparent that various modifications and variations can be effected by a person of sufficient skill in the field. For example and though the vibratory units discussed herein pertain to an inflatable prosthetic device, it will be readily apparent that non-inflatable (i.e., malleable) versions, such as those described in U.S. Pat. Nos. 3,987,789 and 4,267,829 can also be employed, the contents of these patents being herein incorporated by reference in their entirety.

1. A penile prosthetic assembly comprising:
at least one cylinder made from a medical grade material that is configured for surgical implantation within the corpora chambers of a penis;
a vibratory unit attached to the at least one cylinder, the vibratory unit including a motor connected to a portable power source; and
a control mechanism to selectively enable the vibratory unit.

2. The penile prosthetic assembly according to claim 1, in which the control mechanism is a remote control unit configured for wireless operation.

3. The penile prosthetic assembly according to claim 1, in which the control mechanism includes an implantable switch.

4. The penile prosthetic assembly according to claim 1, further comprising:
a pump; and
a fluid reservoir fluidically connected to the pump and the at least one cylinder to enable inflation thereof.

5. The penile prosthetic assembly according to claim 4, wherein the control mechanism selectively controls operation of each of the pump and the vibratory unit to control inflation of the cylinders and vibration thereof.

6. The penile prosthetic assembly according to claim 1, wherein the control mechanism is enabled to provide multiple vibratory modes.

7. A penile prosthetic assembly comprising:
a pump;
a reservoir containing a volume of fluid;
a set of cylinders fluidically connected to the pump and reservoir, the cylinders, pump and reservoir being sized for implantation within the penis of a patient and configured to permit fluid to be selectively pumped into and out of the cylinders for inflation and deflation thereof;
a vibratory unit attached to the set of cylinders;
a portable power source connected to the pump and the vibratory unit; and
a control mechanism for selectively controlling operation of the vibratory unit and the pump.

8. The penile prosthetic assembly according to claim 7, wherein the control mechanism includes an implantable switch accessible to the patient.

9. The penile prosthetic assembly according to claim 7, wherein the control mechanism includes a remote control unit for selectively energizing and deenergizing the vibratory unit(s).

10. The penile prosthetic assembly according to claim 7, in which the control mechanism enables a plurality of vibratory modes.

11. The penile prosthetic assembly according to claim 9, wherein the remote control unit is wirelessly connected to the pump and vibratory unit(s).
